(19) United States
(12) Patent Application Publication
(10) Pub. No.: US 2004/0197265 A1
(43) Pub. Date: Oct. 7, 2004

(54) Methods and pharmaceuticals for treating muscle insulin resistance and related conditions

(76) Inventors: Michael Clark, Tasmania (AU); Stephen Rattigan, Tasmania (AU)

Correspondence Address: NIXON & VANDERHYE, PC, 1100 N GLEBE ROAD, 8TH FLOOR, ARLINGTON, VA, 22201-4714, US

(21) Appl. No.: 10/480,444

(22) PCT Filed: Jun. 11, 2002
(86) PCT No.: PCT/AU02/00752

(30) Foreign Application Priority Data
Jun. 19, 2001 (AU) PR5768

(51) Int. Cl. A61K 49/00

(52) U.S. Cl. 424/9.2; 424/9.52

(57) ABSTRACT

The present invention relates to methods and drugs for ameliorating insulin resistance in skeletal muscle, a major contributing abnormality to impaired glucose handling in such diseases as type 1 and type 2 diabetes, hypertension, obesity and critical care patients. This invention provides drugs, methods for screening those drugs and methods specifically targeted to ameliorating insulin resistance by increasing capillary blood flow in muscle.

### FIELD OF THE INVENTION

The present invention relates to new methods and drugs for ameliorating insulin resistance in skeletal muscle, a major contributing abnormality to impaired glucose handling in such diseases as type 1 and 2 diabetes, hypertension, obesity and critical care patients. A number of drugs currently in use modify insulin release and/or insulin action, which may include the skeletal muscle but none specifically acts to improve muscle capillary blood flow in the immediate sense. This invention provides a new series of drugs and methods specifically targeted to ameliorating insulin resistance by increasing capillary blood flow in muscle. The central tenet is that by so acting, access for insulin and all nutrients is enhanced.

### BACKGROUND OF THE INVENTION

Approximately 80% of the post-absorptive glucose that enters the blood stream from a meal is taken up by the muscles. The rise in blood glucose in the post-absorptive state triggers the release of insulin from the pancreas and this acts on both the liver (to suppress glucose output) and skeletal muscle (to enhance glucose uptake). A reduced ability of the muscle to respond to insulin constitutes insulin resistance for this tissue and because so much of the post-absorptive glucose is intended for muscle, the blood glucose level rises. An immediate effect of the hyperglycaemia is further stimulus of the pancreas to release more insulin and so hyperinsulinaemia can also occur. As time progresses and if left untreated, sequelae develop, including small and large vessel disease. The pancreas may become exhausted giving rise to type 2 diabetes. Insulin resistance in muscle may also have its origins from low physical activity and/or over-eating (obesity), stress (hypertension and critical care patients) or excessive lipid levels in the blood (hyperlipidaemias).

Most researchers currently regard insulin resistance to be the result of impaired insulin signalling or impaired glucose transport (abnormalities in GLUT4 translocation) in the myocytes that constitute the muscle fibres. Only a few research groups support the notion that delivery of insulin and glucose to the myocytes is rate-limiting and their support is based on a key role for total blood flow (see below). The absence of techniques for determining changes in capillary recruitment as these relate to insulin action in normal healthy individuals and impairment in insulin resistant states has prevented other researchers from becoming aware of the key role of capillary recruitment.

Techniques for measuring limb blood (or total blood) flow in vivo that give reproducible results have been available since 1990. Thus, prior to the applicants' work interest in the general area of insulin/glucose delivery to muscle has largely focused on the role of total blood flow to limbs. A number of laboratories have reported an effect of insulin to increase total blood flow to muscle and that this effect is impaired in states of insulin resistance (1-3). However, the role of the increase in total blood flow mediated by insulin is controversial. Several research groups claim that insulin-mediated changes in total blood flow relate poorly to muscle glucose uptake under a number of circumstances, including insulin dose and time course (4-6). In addition, there have been studies where total flow changes persist when glucose uptake is inhibited (7,8). Also, most vasodilators that augment total blood flow to the limbs do not enhance insulin action nor do they overcome insulin resistance (9,10).

The applicants have conducted research in developing new techniques specifically for the measurement of changes in nutritive capillary blood flow in muscle. The idea for these methods grew out of a series of studies using the perfused rat hindlimb where it was established that a tight link between the proportion of nutritive/non-nutritive blood flow in skeletal muscle and metabolism as well as between the proportion of flow and exercise performance exists. From those studies it was realised that hormone and nutrient access was a central process in controlling both muscle metabolism and function. It has been shown that restriction of insulin and glucose access by pharmacologically manipulating flow to be predominantly non-nutritive, created a state of insulin resistance. This was an important observation because it illustrated the marked effect that reduced access for hormone and substrate could play in controlling down-stream metabolism. The search then began for a method, or methods that could detect changes in the proportion of nutritive (capillary) to non-nutritive blood flow in this tissue that might have application in vivo and ultimately to humans. Marker enzymes located in one or other of the two vascular networks (nutritive or non-nutritive) were to provide the key. Thus the first method involved 1-methylxanthine (1-MX), as an exogenous substrate for xanthine oxidase, an enzyme shown by others to reside predominantly in capillary (nutritive) endothelial cells (11). 1-MX was infused intra-arterially and its metabolite 1-methyl urate measured in venous blood by HPLC. Since there was no uptake by the tissue of either the substrate or the product, the extent of conversion was a reflection of capillary exposure. Characterisation under a number of conditions revealed that 1-MX metabolism was indeed directly proportional to nutritive, or capillary flow, which in the constant-flow perfused hindlimb system could be altered by applying various vasoconstrictors or by simulating exercise (12,13). The 1-MX method was tested in vivo using the hyperinsulinaemic euglycaemic clamp in rats and it has been shown for the first time that insulin acted directly to recruit capillary flow in muscle (14) and that pharmacological manipulation to decrease the proportion of nutritive blood flow by an infused vasoconstrictor, created a state of insulin resistance (15). These latter findings directly linked blood pressure through blood redistribution to insulin resistance in vivo a situation that has been reported from a number of epidemiological studies of human populations in the past. In addition, a close link between capillary recruitment and muscle glucose uptake began to emerge from these and previous 1-MX studies.

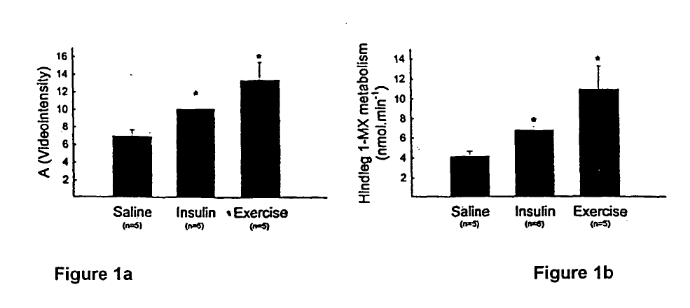

A second method was devised using the latest technologies in ultrasound. The ultrasound method relies on the increased echogenicity of albumin microbubbles which are continuously infused intravenously during data acquisition. The acoustic signal that is generated from the microbubbles when exposed to ultrasound produces tissue opacification which is proportional to the number of microbubbles within the ultrasound beam. Using harmonic pulsing methods essentially all microbubbles within the ultrasound beam are destroyed in response to a single pulse of high-energy ultrasound and an image is obtained. In the time interval between subsequent pulsing episodes, microbubbles flowing into the tissue are replenished within the beam and affect the intensity of the signal from the next high energy pulse. Repeating this process with pulse delays between 50 msec and 20 sec, the beam will be fully replenished and further increases in the time between each pulsing interval will not produce a change to tissue opacification. The rate of microbubble reappearance within the ultrasound beam provides an indication of capillary velocity and the degree of tissue opacification provides a measurement of capillary blood volume (CBV). Images are background-subtracted from images from a pulsing interval of 1000 ms which represents the replenishment of arteries and arterioles thus providing a measurement of capillary flow. The plateau tissue opacification (measured as videointensity) is the determination of capillary blood volume. Using this approach, changes in capillary blood volume in response to insulin and exercise have recently been assessed in the skeletal muscle of the rat hindlimb in vivo and compared to data obtained using 1-MX metabolism (ref 16; FIG. 1). FIG. 1. Comparison of the effect of saline, insulin (3 mU/min/kg, euglycemic clamp ×120 min) or muscle contraction (2 Hz, 1 ms duration, monophasic square waves ×10 min) on capillary blood volume as measured by microbubble videointensity using contrast enhanced ultrasound (FIG. 1a) or the hindlimb extraction of 1-MX (FIG. 1b) measured under identical conditions. Values are means±SE. *, significantly different from saline. Compared to baseline values, saline-infusion resulted in little change in capillary blood volume whereas marked increases in capillary blood volume occurred during euglycemic insulin clamp (3 mU/min/kg), or exercise. This is particularly important as it shows that insulin has an exercise-like effect to recruit capillary blood flow. Exercise is regarded by most physiologists as a “bench-mark” stimulus for capillary recruitment. In addition, FIG. 1 shows that that CEU data correlates well with 1-MX metabolism data. A particular advantage of the ultrasound method is that it is relatively non-invasive and is suitable for human use (17). This opens up possibilities for its use in diagnosis in terms of impaired capillary recruitment in response to insulin and the monitoring of outcomes from therapeutic interventions that might act by increasing capillary recruitment.

The third approach is laser Doppler flowmetry (LDF), where this has already been used for a number of years to study skin blood flow. The applicants have determined that the signal strength from relatively large probes (800 μm), when measured over muscle, directly related to the extent of nutritive flow in the constant flow perfused rat hindlimb. Thus vasoconstrictors that increase metabolism in this preparation increase the LDF signal (18). Conversely, vasoconstrictors that decrease metabolism, also decrease LDF signal (18). Importantly, when under the euglycemic hyperinsulinemic clamp in vivo the laser Doppler signal increased coincident with insulin-mediated increases in glucose infusion (19). Again, this would appear to confirm findings with 1-MX and CEU that insulin mediates a marked capillary recruitment in rat muscle as part of its action in vivo.

The applicant's findings show the following:

Firstly, insulin-mediated capillary recruitment occurs within 5-10 minutes after the commencement of insulin infusion in vivo (20) and is thus an early event.

Secondly, the capillary recruitment mediated by insulin occurs at physiological levels of insulin both in rats and human forearm. When supra-physiological doses of insulin are used there is an increase in total blood flow to the muscles, but this occurs after the increase in capillary recruitment. It appears possible, that the increase in total blood flow to muscle is the result of capillary recruitment. That the increase in capillary recruitment due to insulin occurs independently of an increase in total blood flow suggests that blood has been redirected from the normutritive route to the nutritive capillary network.

Thirdly, blockade of the insulin-mediated capillary recruitment in vivo by either pharmacological manipulation to recruit predominantly non-nutritive blood flow (15), or by treatment of the rats with the inflammatory cytokine, TNFα ((21), led to marked insulin resistance with approx. 50% of the muscle glucose blocked. These findings strongly suggest that insulin-mediated capillary recruitment which increases insulin and glucose access to the myocytes, accounts for about half of the insulin-mediated glucose uptake by muscle in vivo.

Fourthly, in the obese Zucker rat and obese human forearm there is marked impairment of insulin-mediated capillary recruitment that accompanies approximately 50% loss of insulin-mediated glucose uptake.

Fifthly, voluntary exercise training of our local strain of rats for a period of two weeks significantly improves both insulin-mediated muscle glucose uptake and capillary recruitment.

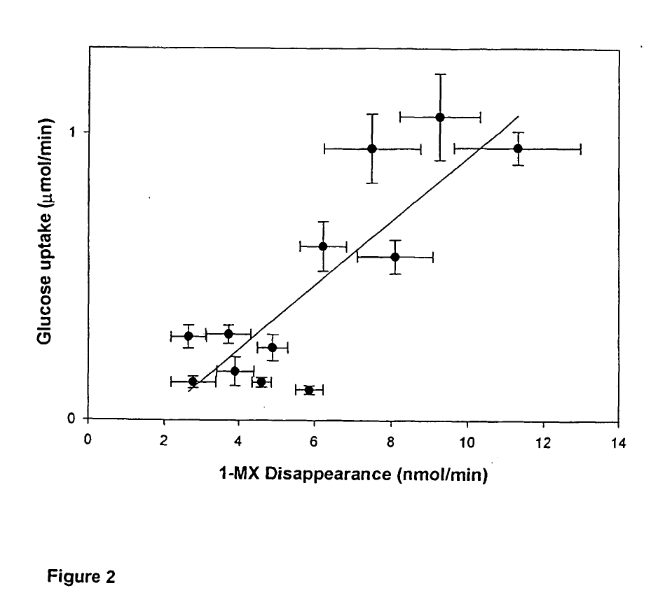

Finally, when all of the data is pooled for the animal studies and muscle glucose uptake is plotted in relation to capillary recruitment a significant correlation is evident (FIG. 2). FIG. 2. Pooled data for in vivo clamps in rats showing correlation between leg glucose uptake and 1-MX disappearance (capillary recruitment).

R²=0.71

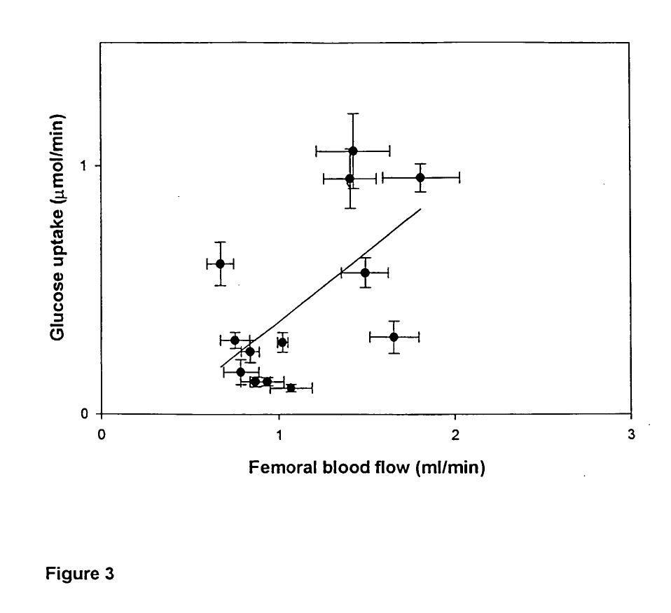

No significant correlation results when muscle glucose uptake is plotted in relation to total limb blood flow (FIG. 3). FIG. 3. Relationship between hindlimb FBF and glucose uptake.

R²=0.37

Accordingly, drugs targeted at increasing muscle capillary blood flow will increase muscle glucose uptake. Moreover, amelioration of an impaired ability of insulin to recruit capillary blood flow in muscle by a new drug will have a significant impact on reversing insulin resistance.

Mechanisms by Which Insulin Acts to Recruit Capillary Blood Flow in Muscle as Indicators to Possible New Drugs Intended to Manipulate This Process.

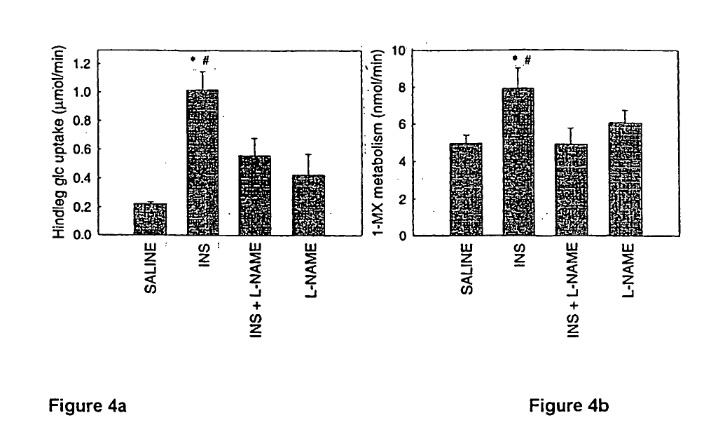

From present knowledge there would appear to be at least three possible mechanisms to account for insulin-mediated capillary recruitment in skeletal muscle. Firstly and most likely, insulin may act at insulin receptors on endothelial cells and an IRS-1/2, P13-K pathway to activate eNOS to produce NO, which in turn permeates adjacent vascular smooth muscle cells to activate soluble guanylyl cyclase and lower the vascular tone of pre-capillary sphincters. In favour of this mechanism is the fact that this process is NO-dependent and is thus consistent with our preliminary data (FIG. 4). There is compelling evidence that insulin acts directly through insulin receptors on endothelial cells to control nutritive capillary flow in skin. Secondly, insulin may act at insulin receptors on the vascular smooth muscle cells (22) via IRS-1/2, P13-K, NOS, cGMP, MBP (myosin bound phosphatase) sequence to cause vasorelaxation. This mechanism would be NO-dependent, free of endothelial cell involvement in signalling and attractive as TNFα is known to inhibit insulin signalling in vascular smooth muscle cells, although to date this has been restricted to the ERK1/2 activation step (23). This mechanism would also lead ultimately to the activation of guanylyl cyclase and the production of cyclic GMP. Thirdly, insulin may act at insulin receptors on skeletal muscle to activate glucose transport and metabolism by the IRS-1/2, P13-K, GLUT4 pathway to produce a metabolite (e.g. adenosine) that permeates adjacent tissue to react with appropriate receptors on endothelial/vascular smooth muscle cells to result in vasorelaxation. This need not involve NO and cyclic GMP, but the applicants have data to show that insulin-mediated capillary recruitment is NO-ependent (FIG. 4). FIG. 4. The effect of L-NAME on insulin mediated increases in hindleg glucose uptake (FIG. 4a) and 1-MX metabolism (FIG. 4b) was examined. Euglycemic clamp conditions (10 mU/min/kg) were conducted for 2h. Values are means±SEM for n=5 for each group. *, significantly different from SALINE. #, significantly different from insulin (INS)+L-NAME. L-NAME also blocked the increase in FBF and raised the mean arterial blood pressure from 100±4 to 125±5 mmHg. This latter mechanism would resemble that occurring in exercise where vasodilatory metabolite(s) are released locally by working muscle to facilitate local blood flow and would be inhibited by agents (e.g. glucosamine) that inhibit muscle glucose metabolism. All three mechanisms should be wortmannin sensitive as P13-K is expected to be involved. A variant of this third mechanism is where a form of NOS is activated in skeletal muscle independently of glucose metabolism. NO could then permeate neighbouring tissue as above. The terminal half of this fourth possible mechanism should be simulated by AMPK activation with AICAR addition (24). Overall, given that the mechanism of capillary recruitment by insulin in rat muscle is NO-dependent (FIG. 4) and that NO acts by producing cyclic GMP, agents that might enhance capillary recruitment by insulin should be targeted to enhance insulin's production of NO or cyclic GMP.

Thus the concept of a new drug(s) that targets muscle capillary blood flow, either acting directly or by enhancing the action of insulin in this respect, where this is impaired in insulin resistance, is the product of the results outlined above. However, there are also parallels to sildenafil (Viagra®), in its capacity to increase blood flow specifically to the corpus cavernosum, and to exercise in causing reactive hyperaemia in working muscles (FIG. 1). Research using the isolated perfused rat hindlimb indicates that when total blood flow does not change, capillary recruitment (or increased nutritive flow) can only occur as a result of flow being switched from the non-nutritive route. Thus the use of a blanket nitrovasodilator, such as nitrprusside etc. is inappropriate. All points in the two vascular routes where tone is maintained are dilated and invariably this favours flow in the route of least intrinsic resistance, which is non-nutritive. A number of research groups have shown that vasodilators (with the possible exception of methacholine), do not increase muscle glucose uptake even though they increase total limb blood flow in vivo, and they do not ameliorate insulin resistance. Novel drugs aimed at increasing nutritive capillary blood flow would act by specifically relaxing sites controlling entry to the nutritive route, and or maintaining or intensifying constriction at sites controlling entry to the non-nutritive route. Exercise is able to achieve this, sildenafil probably does not as a non-nutritive route probably does not contribute in a major way to the blood supply of the corpus cavernosum.

### SUMMARY OF THE INVENTION

In a first aspect the invention provides a method of screening compounds for the ability to increase capillary blood flow, the method comprising:

(a) taking a first measurement of capillary blood flow in a subject;

(b) administering a compound to said subject;

(c) taking a second measurement of capillary blood flow in said subject, and

(d) comparing said first and second measurements,

wherein a positive difference between said first and second measurements indicate the ability of said compound to increase capillary blood flow.

The measurements of capillary blood flow preferably comprise:

administering an ultrasound contrast medium to said subject such that said contrast medium reaches the microvascular capillaries in said subject measuring microvascular capillary blood flow volumes and/or microvascular flow velocity index of said capillaries;

applying a defined signal to said subject;

measuring changes to said microvascular capillary flow where in the measurement is made by ultrasound imaging.

The administration step above may be preceded by administration of insulin.

The defined signal may include any signal potentially or actually capable of affecting microvascular capillary flow.

The screening, when applied to a human subject, may be preceded with a similar screening in another animal of similar biochemistry to the human, for example a rat, so as to minimise unnecessary testing on humans.

As an alternative to the above steps (a) to (d) a 1-MX assay could be used.

The compounds most useful is treating insulin resistance would form the basis of active ingredients in drugs for treating insulin resistance in patients.

In another aspect the invention provides a diagnostic method of tracing microvascular capillary flow response by using the above screening method or capillary flow method in steps (a) to (d) thereby allowing the impact of an agent or compound on said capillary flow to be determined.

In another aspect the invention provides a method of ameliorating the symptoms of insulin resistance in skeletal muscle comprising the administration to said muscle of a drug adapted to improve (increase) insulin—mediated capillary recruitment therein.

The drug may take any physiologically acceptable form and is most preferably administered in conjunction with insulin. The insulin may be derived endogenously or exogenously.

The drug may act acutely, that is within the same time course as insulin, to increase insulin access in real time along with an increase in access of nutrients to myocytes as a result of the recruitment of capillary blood flow.

The drug may act chronically to alter gene expression in a manner such that after several days or weeks of administration of the drug the subsequent ability of insulin to recruit capillary blood flow is improved.

The drug may also be adapted to inhibit cyclic GMP breakdown in terminal arterioles-controlling blood flow to nutritive capillaries.

The drug may also be adapted to enhance production of NO at the same sites as those stimulated by insulin, immediately proximal to the terminal arterioles controlling blood flow to the nutritive capillaries.

The drug may also be adapted to increase muscle glucose metabolism to provide vasodilators that increase NO to dilate the terminal arterioles controlling blood flow to the nutritive capillaries.

The drug may also be adapted to alter gene expression including induction and/or repression of enzyme systems involved with production of NO in endothelial cells.

The drug may also enhance focal production of NO and/or endogenous vasodilators.

The drug may also act on site-specific delivery of micro-encapsulated nitrovasodilator.

The drug may act by blocking blood substances affecting the ability of insulin to recruit capillary flow.

The drug may act via a central mechanism to modify vasomotor neural output.

In another aspect the invention provides a drug screened in accordance with the above method, particularly when used to ameliorate the symptoms of insulin resistance including diabetes, types 1 and 2, hypertension, obesity and critical care patients.

In another aspect the invention provides the above drugs when used in conjunction with insulin.

### DETAILED DESCRIPTION OF THE INVENTION

In order that the nature of the present invention may be more clearly understood preferred forms thereof will now be described with reference to the following non-limiting examples.

Methods for Detecting Insulin-Mediated Capillary Recruitment and Therefore the Means by Which the Present Invention Acts.

In laboratory rats three methods have been used by the applicants to demonstrate that insulin acts to recruit muscle capillary blood flow as part of its normal action in vivo. These are 1-MX (14), CEU/microbubbles (FIG. 1) and LDF (19). Only LDF has been used before by other researchers for this purpose but this has been to assess capillary blood flow changes in human skin. The other two methods 1-MX and CEU/microbubbles are unique to the applicants' and the subject of co-pending applications.

In humans, at this point in time, the CEU/microbubbles method can be used and approval has been granted for use of 1-MX in humans by the Danish authorities. The applicants and their collaborators are applying for wider approval to infuse 1-MX in humans so that the 1-MX method can be used generally.

The 1-MX Method

In principle, 1-methylxanthine (1-MX) is an exogenous substrate for an enzyme located predominantly in the nutritive capillary endothelial cells (much less so in the non-nutritive route and myocytes). Consequently, passage of blood borne 1-MX through the nutritive vascular route leads to its conversion to the product 1-methylurate (1-MU). Chromatographic analysis of arterial and venous samples for 1-MX and 1-MU together with the total blood flow rate over the muscle bed allows the calculation of 1-MX metabolism. A number of our studies using the perfused rat hindlimb have shown tight correlation between nutritive flow (or the proportion of nutritive/non-nutritive flow) and 1-MX metabolism (12,13).

From in vivo studies in rats using the hyperinsulinaemic euglycaemic clamp the applicants have shown that insulin acts to recruit capillary flow in muscle (14). Deliberate impairment of capillary recruitment in an animal model gives rise to insulin resistance (15). At least one model of muscle insulin resistance in animals shows impaired insulin-mediated capillary recruitment (Zucker rat, unpublished). Exercise-training which is beneficial in treating and preventing muscle insulin resistance leads to enhanced insulin-mediated capillary recruitment (unpublished).

Contrast Enhanced Ultrasound/Microbubbles (CEU) Method

The ultrasound method relies on the increased echogenicity of albumin microbubbles that are continuously infused intravenously during data acquisition. The acoustic signal that is generated from the microbubbles when exposed to ultrasound produces tissue opacification that is proportional to the number of microbubbles within the ultrasound beam. Using harmonic pulsing methods essentially all microbubbles within the ultrasound beam are destroyed in response to a single pulse of high-energy ultrasound and an image is obtained. In the time interval between subsequent pulsing episodes, microbubbles flowing into the tissue are replenished within the beam and affect the intensity of the signal from the next high-energy pulse. Repeating this process with pulse delays between 50 msec and 20 sec, the beam will be fully replenished and further increases in the time between each pulsing interval will not produce a change to tissue opacification. The rate of microbubble reappearance within the ultrasound beam provides an indication of capillary velocity and the degree of tissue opacification provides a measurement of capillary blood volume (CBV or MW).

Images are background-subtracted from images from a pulsing interval of 1000 ms which represents the replenishment of arteries and arterioles thus providing a measurement of capillary flow. The plateau tissue opacification (measured as videointensity) is the determination of capillary blood volume. Using this approach, changes in capillary blood volume in response to insulin and exercise have recently been assessed in the skeletal muscle of the rat hindlimb in vivo and compared to data obtained using 1-MX metabolism (16; FIG. 1). Compared to baseline values, saline-infusion resulted in little change in capillary blood volume whereas marked increases in capillary blood volume occurred during euglycemic insulin clamp (3 mU/min/kg). Recent studies have demonstrated that CEU data correlates well with 1-MX metabolism data, and that capillary blood volume increases-2-3 fold during these physiologic doses of insulin (16). A particular advantage of the ultrasound method is that it is relatively non-invasive and is suitable for human use (17).

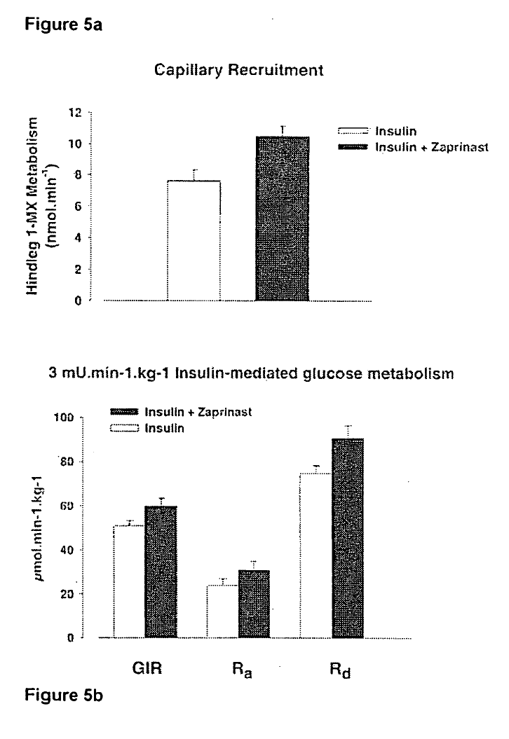

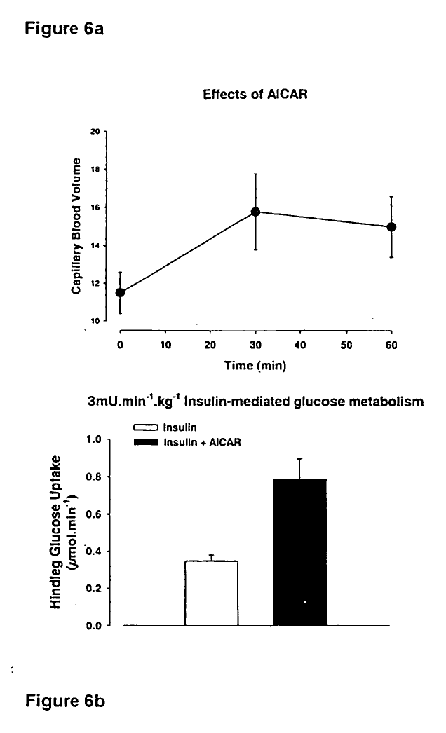

Assay of new drugs acting to increase capillary recruitment in the presence of endogenous or exogenous insulin. This is done in an optional two tier manner, firstly in anaesthetized rats using the hyperinsulinaemic euglycaemic clamp (14) and secondly, in human forearm using a localized hyperinsulinaemic euglycaemic clamp (17). The initial testing in rats is optional, but allows rapid identification of those agents likely to be effective in humans. Typically the means of assay in rats would involve infusion of a physiological dose of insulin that is sub-maximal (e.g. 3 mU/min/kg body weight) in animals that are instrumented to allow continuous monitoring of blood pressure, heart rate and femoral arterial blood flow. The drug to be tested would be infused commencing 1 hour before the infusion of insulin. Arterial blood samples will be taken for glucose analyses in order to check that if the drug increases glucose disposal without insulin infusion. Either way and within 10 minutes of commencing the infusion of the insulin, glucose infusion would be commenced. By assaying arterial blood samples every 15 minutes, the glucose infusion is adjusted to maintain euglycaemia (i.e. 5 mM). In the second hour of the 2 hour clamp markers for muscle glucose uptake (radiolabelled 2-deoxyglucose) and capillary recruitment (1-MX) are infused. At the end of the clamp, arterial and femoral vein blood samples are taken from which capillary recruitment and leg glucose uptake can be calculated from glucose and 1-MX values respectively. Muscles of the lower leg are also removed and the radioactivity therein used to calculate muscle specific glucose uptake. A drug enhancing insulin's action to increase muscle glucose uptake would be expected to increase each of the following: glucose infusion to maintain euglycaemia, leg glucose uptake, muscle specific glucose uptake, and capillary recruitment as indicated by increased 1-MX metabolism (or disappearance). Data for two founder drugs, zaprinast [1,4-dihydro-5-(2-propoxyphenyl)-7H-1,2,3-triazolo(4,5-d)pyrimidin-7-one] and AICAR [5-aminoimidazole-4-carboxamide-1-β-D-ribofuranoside] are shown in FIGS. 5 and 6, respectively. Zaprinast significantly (P<0.05) enhanced insulin-mediated capillary recruitment (1-MX metabolism), glucose appearance (Ra) and glucose disposal (Rd) (FIG. 5). FIG. 5. In this study zaprinast is infused into anaesthetised rats in conjunction with a sub maximal physiologic dose of insulin. FIG. 5a shows that zaprinast enhances insulin-mediated capillary recruitment as indicated by hindleg 1-MX metabolism. Zaprinast also enhanced insulin-mediated glucose appearance (Ra) and most importantly, glucose disposal (Rd). These changes were statistically significant with P<0.05.AICAR recruited capillary flow on its own and when added with insulin, markedly enhanced hindleg glucose uptake (FIG. 6). FIG. 6. In this study AICAR was infused into anaesthetised rats either alone (FIG. 6a) or with insulin (FIG. 6b). AICAR increased capillary recruitment as indicated by CEU and enhanced insulin-mediated hindleg glucose uptake (P<0.05). Drugs of greatest interest will be those that ameliorate insulin resistance in any one of a number of insulin resistant animal models. These might include the genetically obese Zucker rat, and the Intralipid®-infused rat.

Second tier testing of those drugs found to act by enhancing insulin-mediated capillary recruitment in rats are to be tested in humans using the forearm clamp and contrast enhanced ultrasound/microbubbles (CEU) (17). The drug, in a form suitable for oral administration, would be taken one to two hours prior to testing. The patient's response would be tested on two occasions, one with and one without drug administration and the two results compared. Typically, in response to low doses of insulin 0.01 to 0.05 mU/min/kg infused locally into the brachial artery, plasma insulin rises by 70-350 pM in blood perfusing forearm muscle with little or no effect on the systemic insulin, glucose, FFA, catecholamines or amino acid concentrations. As a result, the isolated effect of local insulin on total blood flow into the arm and glucose balance across the arm can be measured. In addition, capillary recruitment in the forearm flexor muscle can be measured using CEU. Total forearm blood flow is measured on the subject by two techniques: capacitance plethysmography and brachial artery ultrasound. For the Doppler flow measurements, an ultrasound system (Sonos 5500, Hewlett-Packard, Andover, Mass.) with a linear-array transducer is used with a transmit frequency of 7.5 MHz to allow 2-D imaging of the brachial artery in the long axis. Brachial artery diameter is measured 2 cm proximal to the tip of the arterial catheter at peak systole using online video calipers. A pulsed-wave Doppler sample blood volume is placed at the same location in the center of the vessel and the mean brachial artery blood velocity measured using on-line angle correction and analysis software. Brachial artery blood flow is calculated from 2-D Doppler ultrasound data using the equation: Q=v□·(d/2)²

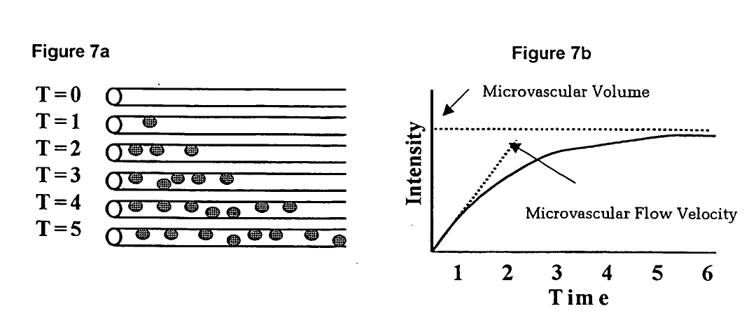

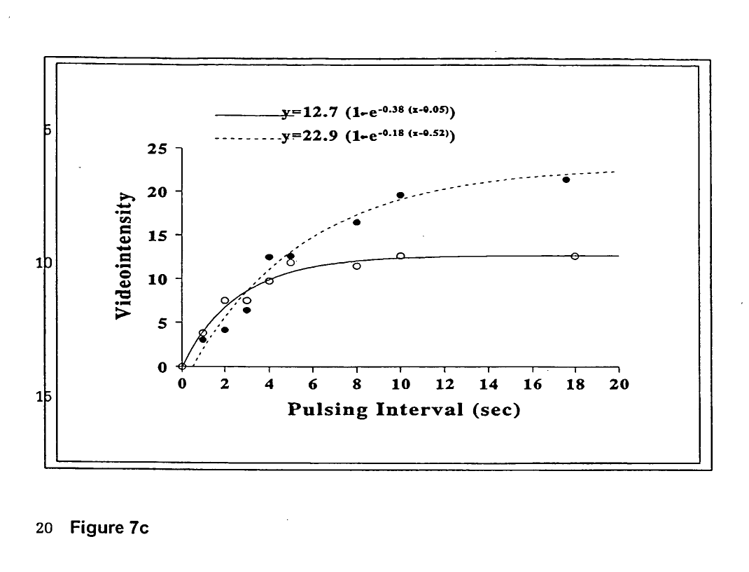

To measure capillary recruitment with CEU, a suspension of albumin microbubbles is infused intravenously in the contra-lateral arm while 2D imaging of the deep flexor muscles of the test forearm is performed. Measurement is made in a trans-axial plane 5 cm distal to be antecubital fossa, using an ultrasound system (Sonos 5500) capable of harmonic imaging. Intermittent imaging is performed with ultrasound transmitted at 1.8 MHz and received at 3.6 MHz. Once the systemic microbubble concentration reaches steady-state (1-1.5 min), intermittent imaging is begun, at pulse intervals ranging from 1 to 15 seconds, thus allowing progressively greater replenishment of the ultrasound beam elevation between destructive pulses. Three images are acquired at each pulse interval. Additional images are acquired with the same beam characteristics at a 30 Hz sampling rate, at which there is replenishment of microbubbles only in vessels with very rapid flow, and these were used as background images. Data are recorded digitally and analyzed using custom-designed software described elsewhere (25). Averaged background frames (acquired at a 30 Hz frame rate) are digitally subtracted from the averaged frames acquired at each pulsing interval. Mean video intensity in the region of interest is measured from the background-subtracted images. Pulsing interval vs. video intensity plots are generated and fitted to an exponential function: y=A(1-e^{□□t}). Where y is the video intensity at a pulsing interval t, A is the plateau video intensity representing microvascular blood volume, and □ is the rate constant reflecting the rate of rise of video intensity (and mean microbubble velocity, or microvascular flow velocity) (FIG. 7) (25,26). FIG. 7. This figure illustrates in more detail how the microvascular blood volume or capillary volume and microvascular flow velocity are determined using CEU. FIG. 7a illustrates the successive filling of a capillary over time after all microbubbles in the capillary have been lysed by a high energy harmonic ultrasound pulse. As the delay time prior to signal detection increases (T0 through T5) the number of microbubbles and hence the videointensity increases. FIG. 7b plots this data for typical signals collected over forearm muscle. The tangent to the upward sloping hyperbolic function is a measure of the rate of microvessel filling (MVFV) while the asymptote that intercepts the y-axis is a measure of the maximal signal seen when the vessels are filled and is determined by the microvascular volume (MW) i.e. capillary volume. In order to derive values for the MVFV and MW, the time versus video intensity plots are fitted to the function: Y=A(1-e^{-βt}), where Y is the video intensity at time t, A is the plateau intensity which represents MW, and p is the time constant of rise and reflects velocity. FIG. 7c shows a typical experiment done before (open circles) and after (filled circles) infusing insulin (3 mU/min/kg) to an anesthetized rat. The plateau videointensity (A) is clearly higher, with no change in the rate of microvascular filling (β).

A positive effect of the drug would be seen as enhancing glucose uptake across the arm and enhanced capillary recruitment typified by an increase in the microvascular volume from CEU over insulin alone. As above, those drugs most useful in treating insulin resistance will be effective in insulin resistant subjects. A positive result in normal healthy individuals is not essential and probably not desirable.

Examples of Drugs Acting to Increase Capillary Recruitment Based on Mechanism.

These may act by inhibiting cyclic GMP degradation in those smooth muscle cells of the terminal arterioles controlling blood flow entry to the nutritive capillaries. As an example, the drug would be targeted to the specific isoenzyme form of cyclic GMP phosphodiesterase expressed in those same smooth muscle cells. The concept for this mechanism is analogous to that accounting for the action of Viagra®.

Alternatively, these drugs may act by altering gene expression over a period of time so that insulin's action to recruit capillary blood flow in muscle is enhanced. A mechanism envisaged here would encompass the induction of enzyme(s) responsible for the production of NO in endothelial cells of the terminal arterioles controlling blood flow entry to the nutritive capillary networks of muscle. Equally, repression of enzyme(s) responsible for NO destruction at these sites, is envisaged. Combined, or separate, such chronic effects of an administered drug would resemble the effects of exercise training as recently reported by us where both insulin-mediated capillary recruitment and muscle glucose uptake was increased (27).

Alternatively, these drugs may act by enhancing focal production of NO in the vicinity of the smooth muscle cells of the terminal arterioles controlling blood flow entry to the nutritive capillaries. The process of enhanced NO production is identical to that normally used by insulin. General or global production of NO in skeletal muscle is counter-productive and would very likely dilate arterioles controlling blood flow to the non-nutritive route.

As a further alternative, these drugs may act by enhancing the focal production of endogenous vasodilators from muscle glucose metabolism. As an example, adenosine is thought to be one of the vasodilators produced by exercising muscle and responsible for the reactive hyperaemia. A logical drug targeted at enhancing the effect of adenosine would act to block adenosine degradation; i.e. an inhibitor of adenosine deaminase.

As a further alternative, these drugs may act using site-specific delivery of a micro-encapsulated nitrovasodilator with the intention of releasing NO in the vicinity of the smooth muscle cells of the terminal arterioles controlling blood flow entry to the nutritive capillaries. There are several enzymes located in the aforementioned specific regions including angiotensin converting enzyme, alkaline phosphatase, and uridine diphosphatase that could be used to hydrolyse polymers constituting the micro-encapsulated nitrovasodilator.

As a further alternative, these drugs may act by blocking substance(s) in the blood that are preventing the normal effect of insulin to recruit capillary flow. For example, we have shown the inflammatory cytokine, TNFα to completely block insulin-mediated capillary recruitment and 50% of the insulin-mediated muscle glucose uptake. It follows that an agent that blocks TNFα would under these circumstances restore normal insulin responses.

Finally, these drugs may act through a central acting mechanism to modify vasomotor neural output thus increasing capillary recruitment by site-specific vasodilatation.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Baron A D, Laakso M, Brechtel G, Edelman S V: Mechanism of insulin resistance in insulin dependent diabetes mellitus: a major role for reduced skeletal muscle blood flow. J. Clin. Endocrinol. Metab. 73:637-643, 1991

2. Baron A D, Steinberg H, Brechtel G, Johnson A: Skeletal muscle blood flow independently modulates insulin-mediated glucose uptake. Am J Physiol 266:E248-E253, 1994

3. Baron A D: Insulin and the vasculature-old actors, new roles. J Investig. Med. 44:406A12, 1996

4. Utriainen J, Malmstrom R, Makimattila S, Yki-Jarvinen H: Methodological aspects, dose-response characteristics and causes of interindividual variation in insulin stimulation of limb blood flow in normal subjects. Diabetologia 38:555-564, 1995

5. Laine H, Yki-Jarvinen H, Kirvela O, Tolvanen J, Raitakari M, Solin O, Haaparanta M, Knnuti J, Nuutila P: Insulin resistance of glucose uptake in skeletal muscle cannot be ameliorated by enhancing endothelium-dependent blood flow in obesity. J. Clin. invest. 101: 1156-1162, 1998

6. Yki-Jarvinen H, Utriainen T: Insulin-induced vasodilatation: physiology or pharmacology? Diabetologia 41:369-379, 1998

7. Scherrer U, Randin D, Vollenweider P, Vollenweider L, Nicod, P: Nitric oxide release accounts for insulin's vascular effects in humans. J Clin. Invest. 94:2511-2515,1994

8. Vollenweider L, Tappy L, Owlya R, Jequier E, Nicod P, Scherrer U: Insulin-induced sympathetic activation and vasodilation in skeletal muscle. Diabetes 44:641-645, 1995

9. Natali A, Bonadonna R, Santoro D, Galvan A, Baldi S, Frascerra S, Palombo C, Ghione S, Ferrannini E. Insulin resistance and vasodilation in essential hypertension. J Clin Invest 94:1570-1576,1994

10. Sarabi M, Lind L, Miligard J, Hanni A, Hagg A, Berne C, Lithell H: Local vasodilatation with metacholine, but not with nitroprusside, increases forearm glucose uptake. Physiol Res 48:291-295, 1999

11. Jarasch E D, Bruder G, Heid H W: Significance of xanthine oxidase in capillary endothelial cells. Acta Physiol. Scand. Suppl. 548:39-46,1986.

12. Rattigan S, Appleby G J, Miller K A, Steen J T, Dora K A, Colquhoun E Q & Clark M G. Serotonin inhibition of 1-methylxanthine metabolism parallels its vasoconstrictor activity and inhibition of oxygen uptake in perfused rat hindlimb. Acta Physiol. Scand. 161: 161-169, 1997.

13. Youd J M, Newman J M B, Clark M G, Appleby G J, Rattigan S, Tong A CY & Vincent M A. Increased metabolism of infused 1-methylxanthine by working muscle. Acta Physiol. Scand. 166: 301-308, 1999.

14. Rattigan S, Clark M G & Barrett E J. Haemodynamic actions of insulin in rat skeletal muscle: evidence for capillary recruitment. Diabetes, 46: 1381-1388,1997.

15. Rattigan S, Clark M G & Barrett E J. Acute vasoconstriction-induced insulin resistance in vivo in rat muscle. Diabetes, 48: 564-569,1999.

16. Vincent M A, Dawson D, Clark A D, Leong-Poi H, Clark M G, Rattigan S & Lindner J R. Physiologic hyperinsulinemia mimics the capillary recruitment induced by exercise in skeletal muscle in vivo. President's selection, LB-15, American Diabetes Association Meeting, San Antonio, USA, June 2000.

17. Coggins M P, Fasy E, Lindner J, Jahn L, Kaul S, Barrett E J. Physiologic hyperinsulinemia increases skeletal muscle microvascular blood volume in healthy humans (Abstract). Diabetes 48:supplement:A220, 1999.

18. Clark A D H, Youd J M, Rattigan S, Barrett E J & Clark M G. Heterogeneity of laser Doppler flowmetry signal in perfused rat muscle indicative of nutritive and non-nutritive. flow. Am. J. Physiol. 280: H1324-H1333, 2001

19. Clark A D H, Barrett E J, Rattigan S, Wallis M G & Clark M G. Insulin stimulates laser Doppler signal by rat muscle in vivo consistent with nutritive flow recruitment. Clin. Sci. 100: 283-290, 2001

20. Vincent M A & Barrett E J. Insulin-induced capillary recruitment precedes changes in skeletal muscl glucose uptake. Abstract to be published in Diabetes, 51 Supplement, 2002

21. Youd J M, Rattigan S & Clark M G. Acute impairment of insulin-mediated capillary recruitment and glucose uptake in rat skeletal muscle in vivo by TNFα. Diabetes, 49: 1904-1909, 2000

22. Kahn A M, Seidel C L, Allen J C, O'Neil R G, Shelat R. Song T: Insulin reduces contraction and intracellular calcium concentration in vascular smooth muscle. Hypertension 22:735-742,1993

23. Goetze S, Kintscher U, Kawano H, Kawano Y, Wakino S, Fleck E, Hseueh, Law R E: Tumor necrosis factor alpha inhibits insulin-induced mitogenic signaling in vascular smooth muscle cells. J Biol Chem 275:18279-18283, 2000

24. Fryer L G, Hajduch E, Rencurel F, Salt I P, Hundal H S, Hardie D G, Carling D: Activation of glucose transport by AMP-activated protein kinase via stimulation of nitric oxide synthase. Diabetes 49:1978-1985, 2000.

25. Wei K, Skyba D M, Firschke C, Jayaweera A R, Lindner J R, Kaul S. Interactions between microbubbles and ultrasound: in vitro and in vivo observations. Journal of the American College of Cardiology 29(5):1081-8,1997.

26. Wei K, Kaul S. Recent advances in myocardial contrast echocardiography. Current Opinion in Cardiology 12(6):539-46,1997

27. Rattigan S, Wallis M G, Youd J M & Clark M G. Exercise training improves insulin-mediated capillary recruitment in association with glucose uptake in rat hindlimb. Diabetes, 50: 2659-2665, 2001.

1. A method of screening compounds for the ability to increase capillary blood flow, the method comprising:
(a) taking a first measurement of capillary blood flow in a subject;
(b) administering a compound to said subject;
(c) taking a second measurement of capillary blood flow in said subject, and
(d) comparing said first and second measurements,
wherein a positive difference between said first and second measurements indicate the ability of said compound to increase capillary blood flow.

2. A method according to claim 1 wherein said measurement of capillary blood flow includes:
administering an ultrasound contrast medium to said subject such that said contrast medium reaches the microvascular capillaries in said subject;
measuring microvascular capillary blood flow volumes and/or microvascular flow velocity index of said capillaries;
applying a defined signal to said subject; and
measuring changes to said microvascular capillary flow.

3. A method according to claim 1 wherein the administration of said compound is preceded by the administration of insulin.

4. A method according to claim 2 wherein said measurement is made by ultrasound imaging.

5. A method according to claim 1 wherein said subject is a human.

6. A method according to claim 5, wherein said human screening is preceded by screening in a non-human animal.

7. A method according to claim 1 wherein steps a) to d) are replaced by an 1-MX assay.

8. A method of treating insulin resistance including the administration of a pharmaceutical composition characterized by an active compound as determined claim 1.

9. A diagnostic method of tracing microvascular capillary flow response including the use of the method of claim 1 to determine the impact of an agent or compound on capillary flow.

10. A method of ameliorating the symptoms of insulin resistance in skeletal muscle comprising the administration to said muscle of a pharmaceutical composition characterized by an active compound as determined by claim 1.

11. A method according to claim 10 wherein said compound is adapted to increase insulin mediated capillary recruitment therein.

12. A method according to claim 10 wherein said pharmaceutical composition is administered in conjunction with insulin.

13. A method according to claim 12 wherein said insulin is derived endogenously or exogenously.

14. A method according to claim 12 wherein said pharmaceutical compound acts acutely within the same time course as insulin.

15. A method according to claim 10 wherein said pharmaceutical composition is also adapted to inhibit cyclic GMP breakdown in terminal arterioles controlling blood flow to nutritive capillaries.

16. A method according to claim 10 wherein said pharmaceutical composition is also adapted to enhance production of NO at the same sites as those stimulated by insulin, immediately proximal to the terminal arterioles controlling blood flow to the nutritive capillaries.

17. A method according to claim 10 wherein said pharmaceutical composition is also adapted to increase muscle glucose metabolism to provide vasodilators that increase NO to delate the terminal arterioles controlling blood flow to the nutritive capillaries.

18. A method of enhancing insulins ability to recruit capillary blood flow in muscles comprising the administration of a pharmaceutical composition characterised by an active compound as determined by claim 1 wherein said composition acts chronically by inducing expression of enzymes responsible for production of NO in endothelial cells of the terminal arterioles controlling blood flow entry to the nutritive capillary network of muscle.

19. A method of enhancing insulins ability to recruit capillary blood flow in muscles comprising the administration of a pharmaceutical composition characterised by an active compound as determined by any claim 1 wherein said composition acts chronically by repressing expression of enzymes responsible for destruction of NO in endothelial cells of the terminal arterioles controlling blood flow entry to the nutritive capillary networks of muscle.

20. A method of enhancing insulins ability to recruit capillary blood flow in muscles comprising the administration of a pharmaceutical composition characterised by an active compound as determined by claim 1 wherein said composition acts either chronically or acutely by enhancing focal production of NO in the vicinity of the smooth muscle cells of the terminal arterioles controlling blood flow entry to the nutritive capillaries.

21. A method of enhancing insulins ability to recruit capillary blood flow in muscles comprising the administration of a pharmaceutical composition characterised by an active compound as determined by claim 1 wherein said composition acts either chronically or acutely by enhancing the focal production of endogenous vasodilators from muscle glucose metabolism.

22. A method according to claim 21 wherein adenosine degradation is blocked.

23. A method of enhancing insulins ability to recruit capillary blood flow in muscles comprising the administration of a pharmaceutical composition characterised by an active compound as determined by claim 1 wherein said composition acts either chronically or acutely by using site-specific delivery of an encapsulated nitrovasodilator with the intention of releasing NO in the vicinity of the smooth muscle cells of the terminal arterioles controlling blood flow entry to the nutritive capillaries.

24. A method according to claim 23 where angiotensin converting enzyme is used to hydrolyse polymers constituting the micro-encapsulated nitrovasodi lator.

25. A method according to claim 23 where uridine diphosphatase is used to hydrolyse polymers constituting the micro-encapsulated nitrovasodilator.

26. A method of enhancing insulins ability to recruit capillary blood flow in muscles comprising the administration of a pharmaceutical composition characterised by an active compound as determined by claim 1 wherein said composition acts either chronically or acutely by blocking substances in the blood that are preventing the normal effect of insulin to recruit capillary flow.

27. A method according to claim 26 wherein a TNFa inhibitor is used to recover insulin-mediated capillary recruitment.

28. A method of enhancing insulins ability to recruit capillary blood flow in muscles comprising the administration of a pharmaceutical composition characterised by an active compound as determined by claim 1 wherein said composition acts either chronically or acutely by acting through a central acting mechanism to modify vasomotor neural output thereby increasing capillary recruitment by site specific vasodilation.

29. A method of improving or increasing capillary blood flow in muscles comprising the administration of a pharmaceutical composition characterised by an active compound as determined by claim 1.

30. A pharmaceutical composition characterised by an active compound screened in accordance with the method of claim 1.

31. A pharmaceutical composition according to claim 20 in combination with insulin.

32. A pharmaceutical composition according to claim 20 wherein said active compound is any one or a combination of [1,4-dihydro-5-(2-propoxyphenyl)-7H-1,2,3-triazolo(4, 5-d)pyrimidin-7-one] and [5-aminoimidazole-4-carboxamide-1-β-D-ribofuranoside].

33. A method according to claim 8 wherein the active compound of said pharmaceutical composition is any one or a combination of [1,4-dihydro-5-(2-propoxyphenyl)-7H-1,2,3-triazolo(4,5-d)pyrimidin-7-one] and [5-aminoimidazole-4-carboxamide-1-β-D-ribofuranoside].

34. A method of ameliorating the symptoms of any one or a combination of insulin resistance including diabetes type 1 and 2, hypertension, obesity and symptoms found in critical care patients comprising the use of a pharmaceutical composition according to claim 30.

35. A method according to claim 1 substantially as hereinbefore described with reference to the examples.

36. A pharmaceutical composition according to claim 30 substantially as hereinbefore described with reference to the examples.
